(19) United States
(12) Patent Application Publication
(10) Pub. No.: US 2004/0180449 A1
(43) Pub. Date: Sep. 16, 2004

(54) Multidimensional chromatography with ion exchange solid phase extraction

(75) Inventors: Alan J. Hamstra, Middleton, WI (US); Lucas D. Roenneburg, Albany, WI (US)

Correspondence Address: PHILIP M. KOLEHMAINEN, GREER, BURNS & CRAIN, LTD., SUITE 2500, 300 SOUTH WACKER DRIVE, CHICAGO, IL, 60606, US

(73) Assignee: Gilson, Inc., Middleton, WI

(21) Appl. No.: 10/386,367

(22) Filed: Mar. 11, 2003

(51) Int. Cl. G01N 1/18

(52) U.S. Cl. 436/178

(57) ABSTRACT

A sample is fractioned prior to chromatographic separation by a multidimensional purification routine including sequential first and second solid phase extraction procedures having different first and second extraction dimensions such as ph and ion strength. The method is automated and performed with a software programmable automated liquid handler system. The first extraction routine produces step graded fractions that are extracted into step graded subfractions in the second extraction routine. The subfractions are separated in an HPLC column with the output columns going to a detector such as a mass spectrometer or a ultra violet detector.

### FIELD OF THE INVENTION

The present invention relates to an improved method for separating or purifying a sample, for example separating peptides or peptide groups from a protein sample.

### DESCRIPTION OF THE PRIOR ART

In the field of proteomics, the current techniques for protein and peptide separations prior to detection by mass spectrometry or other methods are skill intensive and/or slow. One known technique is the use of two dimensional gels in which a linear sample array is migrated sequentially in two steps across the gel using two different techniques such as electrical current separation in one dimension and a salt gradient in the second dimension. The array of separated fractions or individual fractions can then be analyzed. A high degree of user skill is required to obtain reliable and consistent results. In addition, the gel process for a typical protein sample requires a great deal of time, typically measured in days. These are serious disadvantages for large scale separation operations.

Two dimensional chromatography has been proposed in an attempt to provide an automated improvement on two dimensional gel separation. In this chromatography process, a sample is separated in a first chromatographic column based on one characteristic, or dimension, and the separated fractions are then individually separated in a second chromatographic column based on a different characteristic, or dimension. While this process can be automated, the necessity for column switching results in complicated plumbing systems, high cost and long fraction preparation times. Peptide mapping requires a high degree of retention time reproducibility. However, moving liquid through multiple valves and using multiple pumping systems adds many variables that can make it difficult and time consuming to achieve satisfactory retention time reproducibility.

### SUMMARY OF THE INVENTION

A primary object of the present invention is to provide an improved high throughput sample separation method. Other objects are to provide a method that is simpler and very much faster than known two dimensional gel and two dimensional chromatography processes; to provide a method that is simple and fast and is readily automated; to provide a method that is flexible and easily adapted to a variety of sample separation dimensions; to provide a method that achieves a high degree of retention time reproducibility; and to provide a sample separation method that overcomes the disadvantages of known methods.

In brief, in accordance with the invention there is provided a method for separating a sample. The method includes performing a multidimensional purification routine on the sample. This routine includes a first solid phase extraction in which the sample is divided into step gradient fractions using a first extraction dimension, and a second solid phase extraction in which at least one extracted step gradient fraction is further subdivided into step gradient subfractions using a second extraction dimension different from the first extraction dimension. At least one of the step gradient subfractions is chromatographically separated into discrete peaks. The discrete peaks are analyzed.

### BRIEF DESCRIPTION OF THE DRAWING

The present invention together with the above and other objects and advantages may best be understood from the following detailed description of the preferred embodiment of the invention illustrated in the drawings, wherein:

FIG. 1 is a block diagram illustrating steps in the method of the present invention;

FIG. 2 is a simplified elevational view of an automated liquid handler based system for performing the method;

FIG. 3 is a simplified and partly schematic view of solid phase extraction apparatus for use in the method;

FIG. 4 is a diagram illustrating steps in a solid phase extraction procedure included in the invention.

FIG. 5 is a chromatogram of a tryptic digest sample used in an example of the method, detected by ultra violet sensing of the output from a C18 reverse phase column;

FIG. 6 is a graph of the pH gradient formed by the fractions resulting from the first dimension solid phase extraction in the example of the method;

FIGS. 7A through 7F are chromatograms of fraction numbers 1, 3, 4, 5, 6 and 7 resulting from the first dimension solid phase extraction in the example of the method, detected by ultra violet sensing of the output from a C18 reverse phase column; and

FIGS. 8A and 8B are chromatograms of two different subfractions resulting from the second dimension solid phase extraction in the example of the method, detected by ultra violet sensing of the output from a C18 reverse phase column

### DESCRIPTION OF THE PREFERRED EMBODIMENT

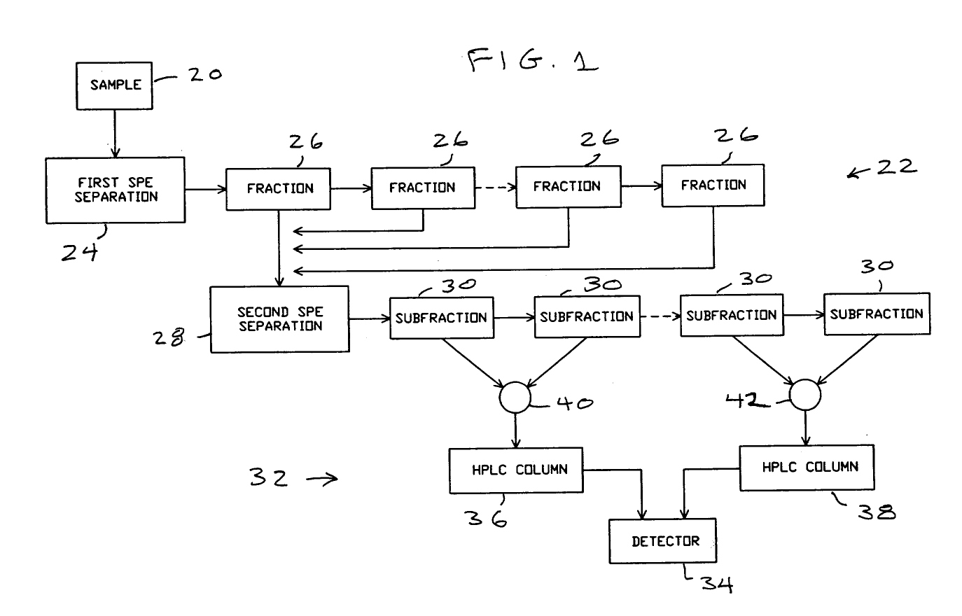

Having reference now to the drawing, and initially to FIG. 1, there is illustrated a sequence of steps of a preferred method in accordance with the present invention. The method separates, or purifies, a sample 20 in a series of procedures that can be automated and performed quickly with relatively simple apparatus without the need for a high degree of skill to obtain consistent and reliable retention time reproducibility.

In the preferred embodiment of the invention, a multidimensional sequence of solid phase extractions is performed in a multidimensional purification routine generally designated as 22 in order to obtain fractions and subtractions of the sample 20. The first step in the routine 22 is to perform a first dimension solid phase extraction procedure 24 in order to obtain a series of fractions 26 from the sample 20. In the next step of the routine 22, a second dimension solid phase extraction procedure 28 is performed upon the fractions 26 in order to obtain a series of subfractions 30 from each fraction 26.

As will appear below, the fractions 26 and the subtractions 30 are step gradient fractions and subtractions. The extraction dimensions of the first and second dimension extractions 24 and 28 are different from one another and each may be any appropriate dimension selected in view of the characteristics of the sample and the results sought. For example, the extraction dimensions may be based on pH, on ion strength, on ion size or on any other known discriminating characteristic. If desired, one or more additional dimensions of solid phase extractions may included in the multidimensional purification routine 22 following the second dimension solid phase extraction procedure 28.

In the method illustrated in FIG. 1, the subtractions 30 resulting from the second dimension solid phase extraction 28 are separated chromatographically in a high pressure liquid chromatography (HPLC) section designated as a whole as 32 The columns from the HPLC section 30 are analyzed with a detector 34.

The high speed of the multidimensional purification routine 22 permits the subtractions 30 to be produced at a rate higher than the throughput rate of a single HPLC column. Accordingly, in the method seen in FIG. 1, the HPLC section 32 includes a pair of HPLC columns 36 and 38 simultaneously processing subfractions 30 in parallel. A pair of injector valves 40 and 42 supply fractions to the columns 36 and 38. Alternatively, the HPLC section 32 could include a single injector valve, and a column switching valve could be used to supply the two HPLC columns 36 and 38. The speed of the multidimensional purification routine 22 could permit the use of more than two simultaneously operating HPLC columns in the HPLC section 32.

Prior to the chromatographic separation in the HPLC section 32, the fractions 30 may be processed in another solid phase procedure, or otherwise processed, for example by being desalted or concentrated or the like.

The detector 32 receives the flowing separated columns from the HPLC columns 34 and 36. The detector 32 may be of any appropriate type depending on the nature of the sample 20 and upon the desired results. Suitable detectors for example are mass spectrometers of various types and ultra violet sensors.

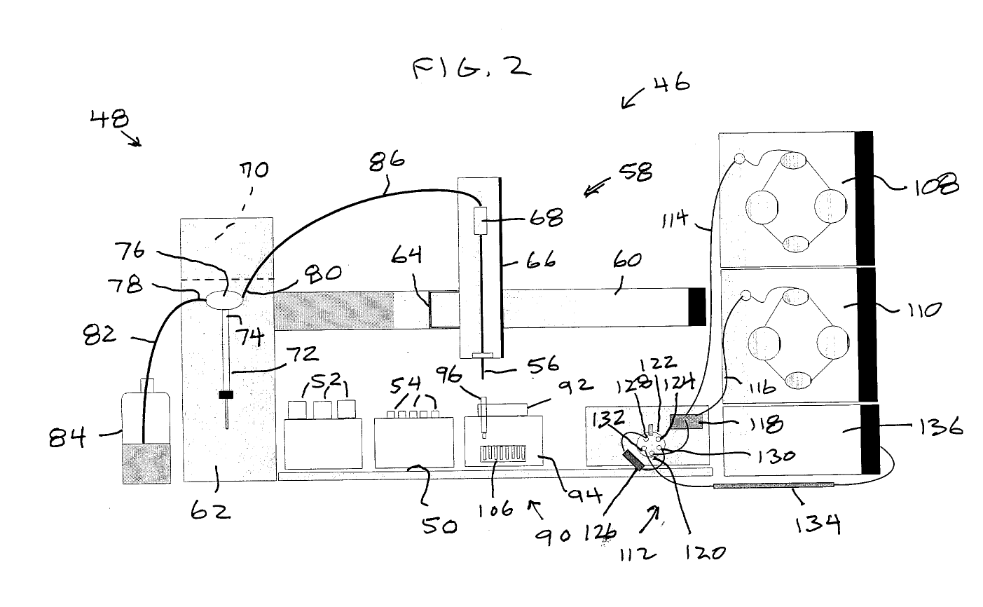

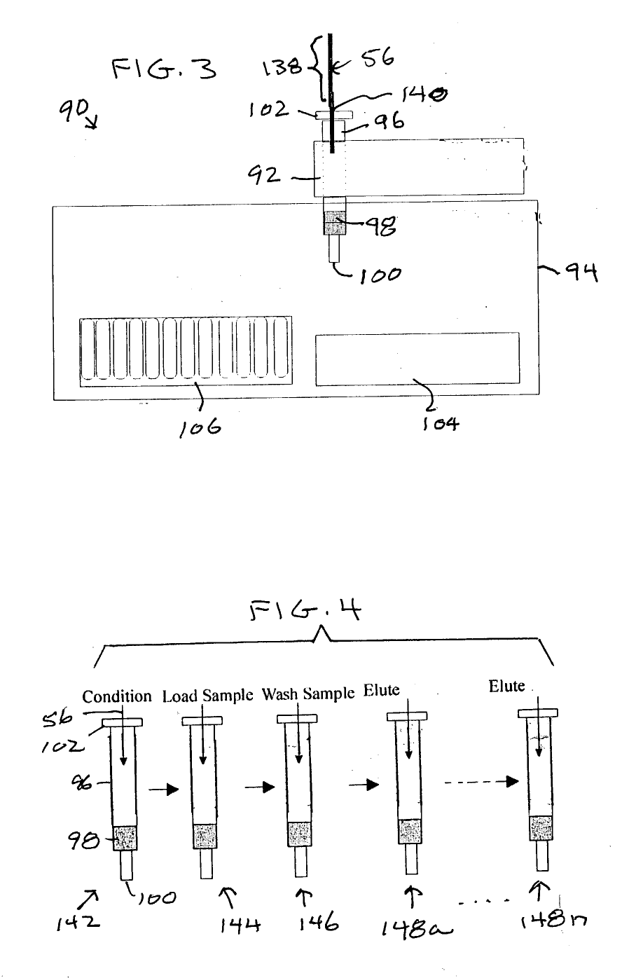

FIGS. 2 and 3 illustrate an apparatus generally designated as 46 for performing the method of the present invention. The apparatus 46 is based upon an automated liquid handler 48 and permits the method to be automated. The liquid handler 48 includes a work surface 50 supporting a number of containers 52 containing buffers and reagents, and other containers 54 for samples 20.

A probe 56 is movable relative to the work surface 50 by an X-Y-Z drive system 58. The drive system 58 includes an X arm 60 extending horizontally from a pedestal section 62, a horizontal Y arm 64 movable in the horizontal X direction along the X arm 60, a vertical Z arm 66 movable in the horizontal Y direction along the Y arm 64, and a probe holder 68 movable in the vertical Z direction along the Z arm 66. A microprocessor based programmable controller 70 in the pedestal 62 operates X, Y and Z drive motors in order to position the probe 56 at any selected position over the work surface 50 and to lower and raise the probe 56 into and out of containers on the work surface 50.

A syringe pump 72 carried on the pedestal 62 is operated by the controller 70 to provide negative or positive pressure to a pump port 74 of a probe valve 76. The valve 76 is operated by the controller 70 to interconnect the pump port 74 with either a rinse port 78 or a probe port 80. The rinse port 78 communicates through a conduit 82 with a supply 84 of a rinse solvent and the probe port 80 communicates through a conduit 86 with the probe 56. With the ports 74 and 80 in communication, negative pressure is applied to the probe 56 by the syringe pump 72 in order to aspirate liquid or air into the probe 56, and positive pressure is applied to the probe 56 in order to discharge liquid or air from the probe 56.

Many different commercially available automated liquid handlers can be used to perform the method of the invention. The automated liquid handler 48 may be of the construction disclosed in Gilson U.S. Pat. No. 4,422,151, incorporated herein by reference. The disclosure of that patent may be referred to for a more detailed description of the liquid handler 48.

In order to perform the solid phase extraction procedures of the invention, the apparatus 46 includes a solid phase extraction (SPE) unit generally designated as 90. The SPE unit 90 includes a rack 92 slideably supporting a tray 94 holding one or more SPE cartridges 96, one of which is seen in FIGS. 2 and 3. Each cartridge 96 holds a body 98 of cartridge packing material providing a solid phase appropriate to the particular solid phase extraction procedure to be performed. The bottom of the cartridge 96 includes a discharge opening 100, and the top is closed by a seal cap 102 having a small opening for receiving the probe 56. When probe 56 is inserted through cap 102 and into the SPE cartridge 96 as seen in FIG. 3, an air tight seal is formed so that the cartridge 92 can be pressurized by the probe 56.

Within the tray 94 are a drain trough 104 and an array 106 of receptacles, preferably a microplate, such as a ninety-six well deep well microtiter plate. The trough 104 and the plate 106 are side by side in the tray 94. The controller 70 moves the probe against the rack 92 in order to slide the rack 92 back and forth on the tray 94 to locate the SPE cartridges 96 either over the trough 104 or to locate one or more specific SPE cartridges directly above corresponding receptacles or wells of the plate 106. Depending upon the sample 20 to be separated, and the number of fractions 26 and subfractions 30 to be extracted, two or more SPE units 90 may be used on the work surface 50.

As seen in FIG. 2, the apparatus 46 includes a pair of high pressure pump units 108 and 110 and an HPLC injector port assembly 112. The pumps 108 and 110 supply mobile phase constituents through conduits 114 and 116 to a mixing section 118 of the injector port assembly 112. The mixing section 118 is connected to a mobile phase inlet 120 of an injector valve 122. The probe 56 delivers liquid to the sample inlet 124 of the valve 122. A sample loop 126 is connected between ports 128 and 130. An outlet port 132 is connected to an HPLC column 134. The loop 126 may be an external pre-column able to perform sample pre-concentration prior to injection into the column 134. The outlet of column 134 is connected to an ultra violet detector 136.

The pumps 108 and 110 and the injector valve 122 are controlled by the controller 70. In a sample loading position of the valve 122, liquid from the probe 56 flows into and is held in the sample loop 126 and the ports 120 and 132 are interconnected so that mobile phase flows at high pressure to the column 134. The valve 122 is operated by the controller 70 to an alternate, sample injection position in which the sample loop 126 is connected between the ports 120 and 132 so that the sample from the loop moves with the mobile phase for separation into the column 134. The separated output from the column 134 flows to the detector 136 where separated peaks are detected. The apparatus 46 may include a second injection port assembly 112 and a second HPLC column as shown in FIG. 1 if desired.

The controller 70 interfaces by way of suitable cabling and/or a common bus with the components of the X-Y-Z drive system 58, the syringe pump 72 and the probe valve 76, the high pressure mobile phase pumps 108 and 110, the injector valve 122 and the detector 136. This permits the method of the invention to be entirely automated so that it is performed at a high throughput rate without requiring a high degree of operator attention or skill. The controller 70, either operating alone or in combination with an additional remote controller such as a remote computer, is software programmable to carry out the steps of the method of the invention.

FIG. 4 illustrates steps in the method that are performed with the apparatus 46 during a solid phase extraction procedure of the multidimensional purification routine 22 of the present invention. FIG. 3 illustrates a subroutine that is repeated with various liquids at points during the procedure. Negative pressure is applied to probe 56 by the syringe pump 72, and the probe 56 aspirates an air gap push volume of air 138 (FIG. 3) followed by a segment of liquid 140 (FIG. 3) from one of the containers 52 or 54 (FIG. 2). The probe 56 is inserted through the cap 102 and into an SPE cartridge 96. A positive pressure is applied to probe 56 by the syringe pump 72 to discharge the liquid segment 140 into the SPE cartridge above the solid phase packing material 98. Continuing positive pressure acting through the air gap 138 within the sealed cartridge 96 forces the liquid 140 into and through the solid phase packing material 98.

In a solid phase extraction routine, as seen in FIG. 4, the tray 92 and an SPE cartridge 96 are initially over the drain trough 104. At step 142 in FIG. 4, the solid phase material 98 is conditioned. The probe 56 aspirates an air gap 138 and a phase conditioning liquid from a selected container 52, and discharges the conditioning liquid into the sealed SPE cartridge where it is forced by pressure into the solid phase material 98. Excess conditioning liquid exits the cartridge discharge opening 100 and falls into the trough 104.

In the next step 144, sample is loaded. The probe 56 aspirates an air gap 138 and a liquid sample from a selected container 54, and discharges the sample liquid into the sealed SPE cartridge 96 where it is forced by pressure into the conditioned solid phase material 98. Any excess sample liquid exits the cartridge discharge opening 100 and falls into the trough 104. In the next step 146, the sample laden solid phase material 98 is washed. The probe 56 aspirates an air gap 138 and a liquid wash solution from a selected container 52, and then discharges the wash liquid into the sealed SPE cartridge 96 where it is forced by pressure into and through the solid phase material 98. The wash liquid exits the cartridge discharge opening 100 and falls into the trough 104.

The extracted samples are then eluted from the SPE cartridge 96 as indicated by steps 148a through 148n for n number of fractions. The eluting liquid may have a suitable step graduated characteristic, differing among fractions. The probe 56 moves the tray 92 along the rack 94 and over the well plate 106 with the discharge opening 100 in alignment with a first individual well. The probe 56 aspirates an air gap 138 and an eluting liquid from a selected container 52, and then discharges the eluting liquid into the sealed SPE cartridge 96 where it is forced by pressure into and through the solid phase material 98. The eluted fraction 26 is received into the aligned well of the plate 106.

The tray 92 is then moved to align the discharge opening 100 of the cartridge 96 with another well in the plate 106. The eluting step is repeated n times to discharge n eluted fractions 26 into n wells of the plate 106 for further processing. In some extraction routines all of the fractions may not be eluted from a single cartridge, and a number of separate cartridges may be used.

Following the first dimension solid phase extraction routine 22, the individual fractions 26 in the wells of plate 106 are processed in the second dimension solid phase extraction 28. The solid phase extraction process as described above is repeated using a solid phase and conditioning, washing and eluting liquids corresponding to the different dimension of the second extraction routine 28. At the conclusion of the second dimension solid phase extraction routine, wells of the well plate 106 contain a large number of subfractions 30. For example, if twelve fractions 26 are extracted in the first dimension solid phase extraction 24, and if five subfractions 30 are extracted from each fraction 26, sixty subfractions 30 will be prepared.

Following the multidimensional purification routine 22, the subtractions 30 may be processed in additional dimensions. In the illustrated embodiment of the invention, the subtractions 30 are sequentially chromatographically processed in the HPLC column 134, with the column output flowing to the ultra violet detector 136 for analysis. Detection could also be performed by mass spectrometry or the like. For chromatographic processing, the probe sequentially transfers subtractions 30 through the injector valve port 124 and into the sample loop 126 from which they are entrained in the mobile phase supplied from the high pressure pump units 108 and 110. The constituents from pumps 108 and 110 differ and their proportions are varied over time to obtain a graded separation in the HPLC column 134. The output from the ultra violet detector 136 is preferably recorded as a series of chromatograms of the sequence of subfractions.

Depending upon the capabilities of the apparatus 46 and automated liquid handler 48, the steps in the method of the invention could be performed at least in part simultaneously rather than in strict sequence. In a liquid handler having independently movable probes, for example, some of the second dimension solid phase extractions could be performed before the first phase solid phase extraction is completed. Also, subtractions from the second dimension solid phase extraction could be forwarded for chromatography before the second dimension solid phase extraction is completed. The simultaneous processing could increase throughput.

### EXAMPLE

In this example the method of the invention is used to separate peptides and peptide groups from a protein sample. The sample 20 is a tryptic digest, specifically of Bovine Serum Albumin (BSA) and Cytochrome C pH 4.0. FIG. 5 is a reverse phase C18 chromatogram of the starting sample material. The peak population density does not provide sufficient separation for effective analysis. The result is greatly improved without substantially added time by the method of the present invention

In the first dimension solid phase extraction, chromatofocusing is used to separate the proteins into step graded fractions according to their iso-electric points. A self-generated pH gradient is formed by first equilibrating the column to a pH of 4.0. The tryptic digest (pH 3.0) is applied. A low concentration buffer at pH 7.0 is applied. A pH gradient is generated as more elution buffer is applied. The first fractions will have pH very similar to starting pH but as more elution buffer is applied fraction pH will begin to move towards the buffer pH.

An SCX cartridge (aromatic sulfonic acid 1 ml/100 mg) is first conditioned with 1 ml volume of 0.5 mM ammonium Acetate buffer pH 4. 1 ml of Tryptic Digest is added and washed with 1 ml of deionized water. 1 ml aliquots of 25 mM ammonium acetate buffer solutions at pH 4.0, 5.0, 6.0, 7.0, and 8.0 are added. Each aliquot is collected into a separate well of a 96 deep well micro titer plate. Fractions are collected until the pH equals that of the elution buffer (about 1 to 2 fractions per buffer solution).

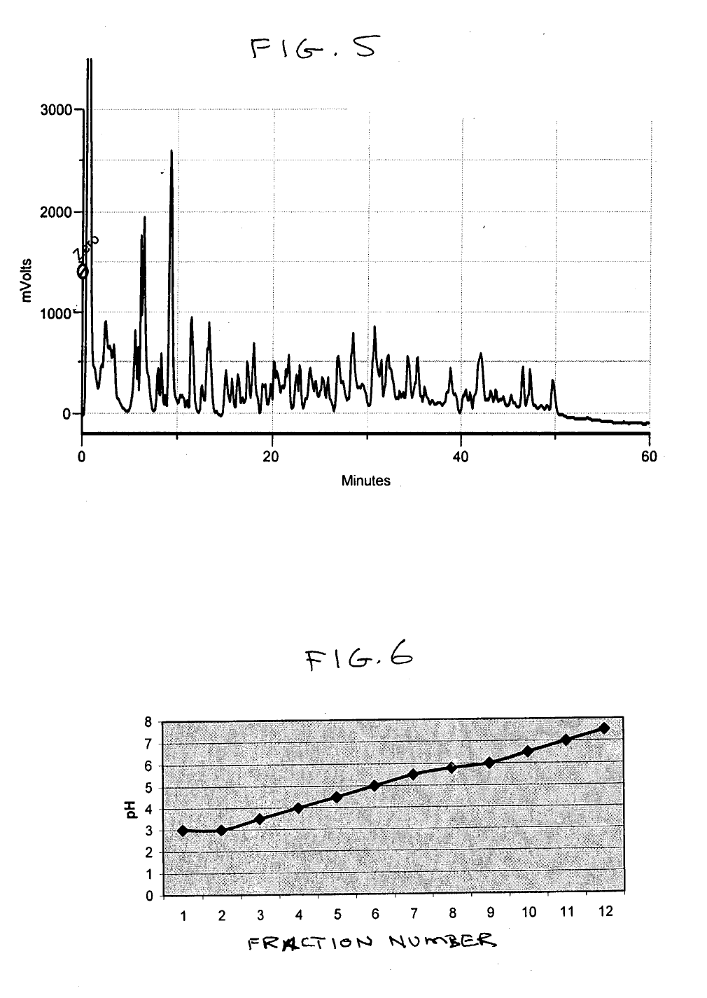

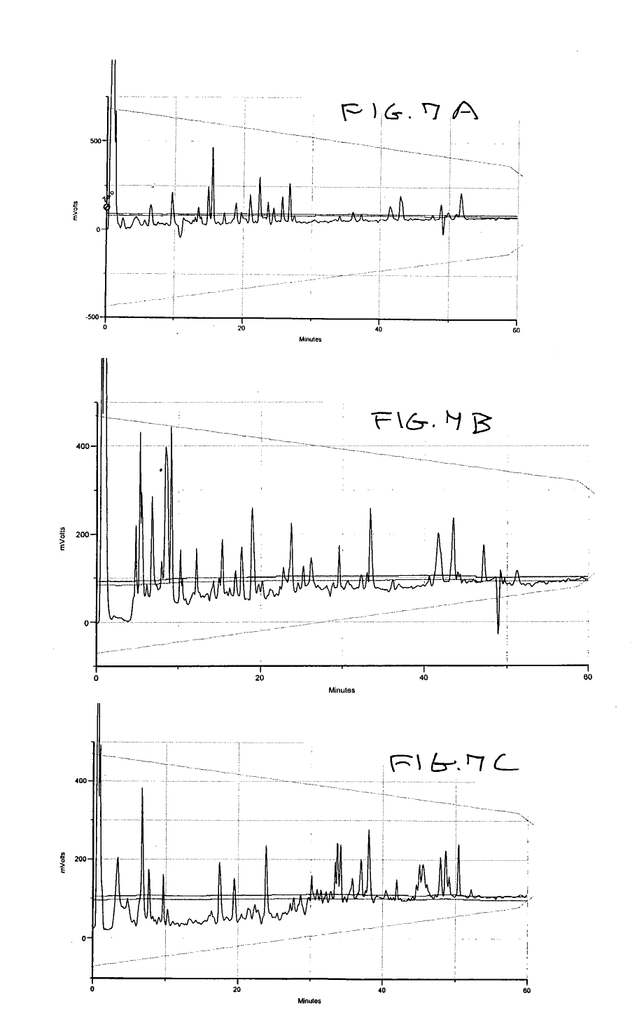

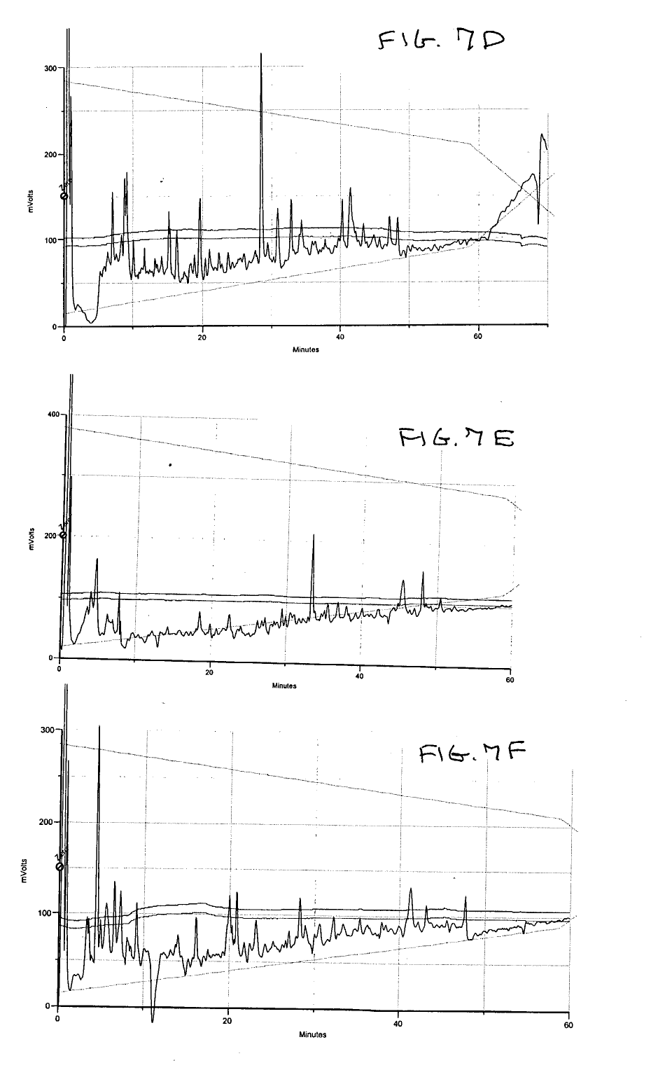

In this example, twelve chromatofocused step graded fractions result from the pH first dimension solid state extraction. FIG. 6 is a graph showing the pH of these twelve fractions. FIGS. 7A through 7F are reverse phase C18 chromatograms of six of these fractions. The diversity of the fractions is apparent.

In the second dimension solid state extraction routine, each chromatofocused fraction is run through an ion exchange separation using buffers of differing ion strength. Each fraction is loaded to a separate SCX ion exchange column. The cartridges are first conditioned with 1 ml 0.5 mM ammonium acetate buffer pH 4.0. The fractions are loaded to the columns and washed with 1 mL deionized water. The subfractions are eluted with a 100 mM and 500 mM ammonium acetate solution. Each buffer concentration subfraction is collected into a separate well.

Other extraction steps could be added without substantially changing the throughput rate. An additional dimension that could be added or used to replace the pH or ion size dimension is an extraction step using size exclusion cartridges.

Following the multidimensional solid phase extractions, The second dimension separated subfraction samples are injected into the HPLC system and run on a reverse phase capillary column (C4, C8, or C18). The HPLC column is a 300 um ID×15 cm column. For the constituents of the mobile phase, pump 108 supplies ninety-five percent H2O, five percent acetonitrile (ACN) with 0.1 percent formic acid, while pump 110 supplies ninety-five percent ACN, five percent H2O, with 0.1 percent formic acid. The reverse phase gradient profile is as follows:

TABLE 1

| | |
|---|---|
| 0 minutes: | Pump A 98%, Pump B 2% Ramped to 35% |
| | pump B over 76 minutes |
| 76 minutes: | Pump A 65%, Pump B 35% Ramped to 75% |
| | pump B over 2 minutes |
| 78 minutes: | Pump A 25% Pump B 75% |
| 88 minutes: | Pump A 25% Pump B 75% Ramped to 98% |
| | Pump A over 1 minute |
| 89 minutes: | Pump A 98%, Pump B 2% |
| 110 minutes: | Pump A 98%, Pump B 2% |

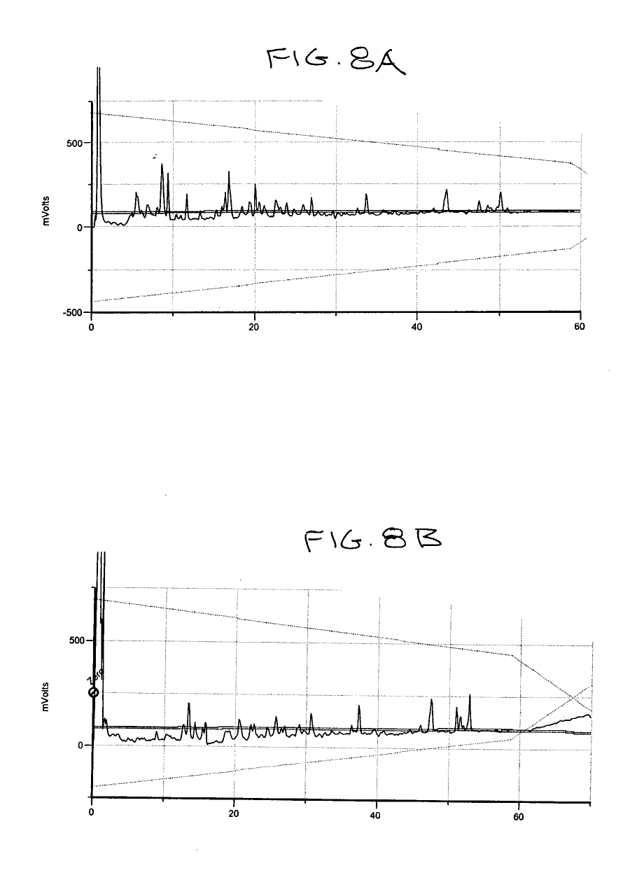

The columns are connected to a UV/Vis detector with a capillary or nano flow cell installed depending on the size of the reverse phase column for verification of separation. The resulting chromatograms of two representative subfractions are seen in FIGS. 8A and 8B. Comparing the large quantity of information that would appear, for example, appears in sixty chromatograms like FIGS. 8A and 8B with the limited information in the original unseparated sample seen in FIG. 5, it can be seen that the sample separation method of the invention provides a substantial improvement, with high throughput rates and good retention time reproducibility.

While the present invention has been described with reference to the details of the embodiment of the invention shown in the drawing, these details are not intended to limit the scope of the invention as claimed in the appended claims.

What is claimed is:

1. A method for separating a sample comprising the steps of:
(a) performing a multidimensional purification routine on the sample, said routine including:
(i) a first solid phase extraction in which the sample is divided into fractions using a first extraction dimension; and
(ii) a second solid phase extraction in which at least one extracted step gradient fraction is subdivided into subfractions using a second extraction dimension different from the first extraction dimension;
(b) chromatographically separating at least one of the step gradient subfractions into discrete peaks; and
(c) analyzing the discrete peaks.

2. The method of claim 1 wherein the sample includes a protein and the fractions, subfractions and discrete peaks are peptide groups or peptides.

3. The method of claim 1 wherein one of the dimensions is pH.

4. The method of claim 1 wherein one of he dimensions is ion strength.

5. The method of claim 1 wherein one of the dimensions is size.

6. The method of claim 1 wherein said extraction steps include sequential displacement of portions of the sample or step gradient fraction into a solid phase.

7. The method of claim 6 wherein said displacement is effected by positive pressure.

8. The method of claim 7 wherein said dimensions are pH and ion strength.

9. The method of claim 8 wherein said separating step includes reverse phase capillary column high pressure liquid chromatography.

10. The method of claim 1 wherein said separating step includes directing a plurality of the step gradient subfractions to a plurality of chromatography columns.

11. The method of claim 1 wherein said analyzing step includes ultra violet sensing of the peaks.

12. The method of claim 1 wherein said analyzing step includes flowing the separated peaks into a mass spectrometer.

13. The method of claim 1 wherein said multidimensional purification routine is automated.

14. The method of claim 13 wherein said multidimensional purification routine is performed with a computer controlled automated liquid handler.

15. The method of claim 1, said first and second solid phase extractions producing step gradient fractions and subfractions.
